(19) United States
(12) Patent Application Publication
(10) Pub. No.: US 2008/0208094 A1
(43) Pub. Date: Aug. 28, 2008

(54) Orthotic Device Having Adjustable Stabilizing Member and Tensioning Arms

(76) Inventor: Eric Lee Gaylord, Matthews, NC (US)

Correspondence Address: SUMMA, ALLAN & ADDITON, P.A., 11610 NORTH COMMUNITY HOUSE ROAD, SUITE 200, CHARLOTTE, NC, 28277, US

(21) Appl. No.: 11/680,093

(22) Filed: Feb. 28, 2007

(51) Int. Cl. A61F 5/00

(52) U.S. Cl. 602/23

(57) ABSTRACT

An orthotic device having an adjustable stabilizing member secured thereto in which the device has a body member for engaging portions of a lower leg and foot, and a flexible sheet member for adjustably and removably securing the device to the leg and foot. Front and rear tensioning arms are provided to adjustably support the leg and foot. The stabilizing member is manually adjustable by a user without requirement of mechanical devices or heat treatment.

### BACKGROUND OF THE INVENTION

This invention relates to orthotic devices for the treatment of plantar fasciitis with support and adjustment features which enhance the comfort, adjustability and support provided by the device. Plantar fasciitis is a medical condition characterized by inflammation of the plantar fascia (i.e., thick tissue on the bottom of the foot) that causes heel pain and disability. More specifically, the plantar fascia is a thick band of tissue that covers the bones on the bottom of the foot and extends from the base of the heel bone to each of the toes of the foot. Plantar fasciitis results when, either suddenly or gradually, motion or lack of arch support causes the flattening of the arch or the spreading of the toes such that sufficient pressure is exerted on the bottom of the foot to tear the plantar fascia. Injury often occurs as the result of hyperextension of the toes in the upward direction during exercise. Plantar fasciitis can be debilitating in that everyday activities such as walking and standing are painful. Typically, most pain is experienced in the heel area, where the plantar fascia attaches to the heel and where tears and separations often occur. Plantar fasciitis is most efficiently treated by wearing a foot support designed to heal the condition while permitting the user to remain relatively mobile.

Some plantar fasciitis supports are designed for use only when the patient is resting (i.e., when little or no weight is applied to the injured foot). Other plantar fasciitis supports, such as the present invention, can be worn by the patient during limited activity (e.g., walking to and from bed) as well as when at rest.

Known supports are often too bulky to be readily worn with footwear, or too flimsy to provide adequate support. Other known supports provide inadequate adjustment or, when provided, the manner of adjustment requires various mechanical devices. The present invention provides an orthotic device for the treatment of plantar fasciitis which addresses these problems.

### SUMMARY OF THE INVENTION

The invention is an orthotic device 10 for a lower leg having a body member 11 that engages portions of the lower leg and foot, a flexible sheet member 15 that secures the body member to the leg and foot, and a manually adjustable stabilizing member 14 fixed to sections of the body member. In particular, upper and lower sections of the stabilizing member 14 and body member 11 are adjustable by a user. By manually adjusting the stabilizing member 14, the user may select a desired angle between the relative positions of the upper and lower sections 12, 13 of the body member 11. Providing a manually adjustable stabilizing member 14 in the body member 11 facilitates the ease of adjusting the desired angle of the foot relative to the leg. Advantageously, the stabilizing member 14 is sufficiently rigid to resist movement during limited activity such as walking from one room of a house to another.

The body member 11 can be formed from plastic while the stabilizing member 14 can be formed from any number of semi-rigid materials such as metal or alloy. The body member 11 may be covered with cloth or similar textile material. The sheet member 15 can be formed from an elastomeric material.

More specifically, the orthotic device of the invention includes a body member 11 having an upper section 12 for engaging an anterior portion of the lower leg and a lower section 13 for engaging a dorsal portion of a foot, a manually adjustable stabilizing member 14 fixed to portions of the upper and lower sections 12, 13 of the body member 11, and a flexible sheet member 15 for adjustably securing the body member against the lower leg and foot. In one embodiment, the flexible sheet member 15 has one portion fixed to an upper section 12 of the body member 11 and another portion removably attachable to the upper section 12 of the body member 11. Further, the flexible sheet member 15 may define at least one opening 23 for receiving portions of a heel.

The orthotic device of the invention may also include a pair of rear or heel tensioning arms 25, 26 and a pair of front or foot tensioning arms 30, 31. The pair of heel tensioning arms 25, 26 is positioned at opposing sides of the opening 23 in the flexible sheet member 15. The pair of foot tensioning arms 30, 31 is positioned at opposite sides of a lower portion of the flexible sheet member 15. The heel and foot tensioning arms 25, 26, 30, 31 are removably attachable to the body member 11 with fasteners 22, thus permitting a user to adjustably secure the device 10 to the lower leg and foot.

In one embodiment, the orthotic device 10 may further provide a pair of flexible panels 24 that connect lower portions of the sheet member 15 to edges of the lower section 13 of the body member 11. As configured, the panels 24 position lower portions of the sheet member 15 against a lower portion of the foot.

The orthotic device may further include a base pad 32 fixed to a lower portion of the flexible sheet member 15 such that the base pad 32 is positioned substantially adjacent to the pair of flexible panels 24. In particular, the base pad 32 is positioned substantially adjacent to the lower section 13 of the body member 11 such that the base pad 32 engages a lower portion of the foot. In this embodiment, the flexible panels 24 promote initial fitment of the device on the foot and the base pad 32 provides cushioning for the lower foot during limited activity.

Thus, the invention provides, among other benefits, an adjustable stabilizing member 14 that permits a user to manually adjust the angle of the upper section 12 of the body member 11 relative to the lower section 13 of the body member, thereby maintaining a desired position of the foot relative to the lower leg during rest and limited activity. Moreover, the invention provides heel and foot tensioning arms 25, 26, 30, 31 that adjustably secure the device to the lower leg and foot of the wearer. The combination of the adjustable stabilizing member 14 and the semi-rigid body member 11 of the invention promotes ease of adjustment and improved support lacking in known orthotic devices.

### BRIEF DESCRIPTION OF THE DRAWINGS

The foregoing and other objects and advantages of the invention and the manner in which the same are accomplished will become clearer based on the following detailed description taken in conjunction with the accompanying drawings in which:

FIG. 1 is a perspective view of one embodiment of the orthotic device of the invention;

FIG. 2 is a front elevation view of one embodiment of the orthotic device;

FIG. 3 is a side elevation view of one embodiment of the orthotic device;

FIG. 4 is a rear elevation view of one embodiment of the orthotic device;

FIG. 5 is a bottom plan view of one embodiment of the orthotic device;

FIG. 6 is a partial cross-sectional view of one embodiment of the orthotic device taken generally along line 6-6 of FIG. 2;

FIG. 7 is a partial cross-sectional view of one embodiment of the orthotic device taken generally along line 7-7 of FIG. 2;

FIG. 8 is a partial cross-sectional view of one embodiment of the orthotic device taken generally along line 8-8 of FIG. 2;

FIG. 9 is a perspective view of one embodiment of the orthotic device;

FIG. 10A is a partial side elevation view of one embodiment of the orthotic device depicting one position of the upper section of the body member relative to the lower section of the body member at angle a; and

FIG. 10B is a partial side elevation view of one embodiment of the orthotic device depicting another position of the upper section of the body member relative to the lower section of the body member at angle a′.

### DETAILED DESCRIPTION OF THE INVENTION

The invention will now be described more fully hereinafter with reference to the accompanying drawings, in which multiple embodiments of the invention are shown. This invention may, however, be embodied in different forms and should not be construed as limited to the embodiments set forth herein. Rather, these embodiments are provided so that this disclosure will be thorough and complete and will fully convey the scope of the invention to those skilled in the art. Like numbers refer to like elements throughout. Further, like numbers with the prime notation refer to like or similar elements of the structure.

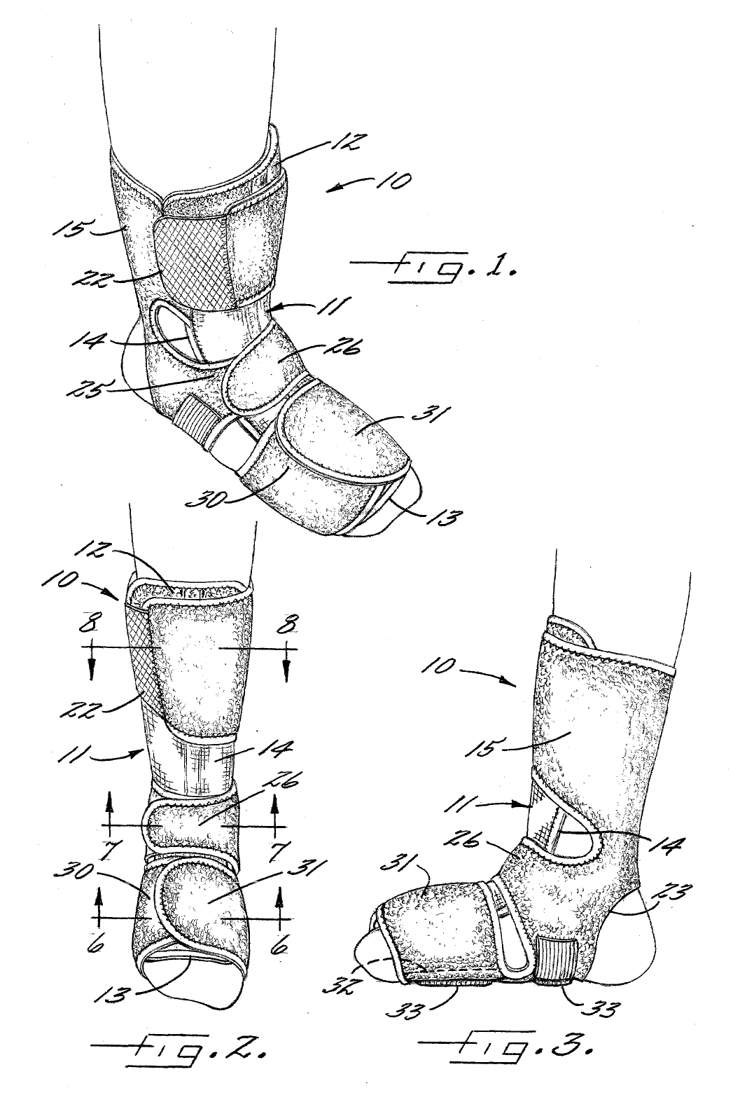

An overall view of an embodiment of an orthotic device of the invention is set forth at 10 in the perspective views of FIGS. 1 and 9. As depicted in FIG. 9, the orthotic device 10 includes a body member 11 having an upper section 12 and a lower section 13, an adjustable stabilizing member 14 fixed to the body member 11, and a flexible sheet member 15 having a portion that is foldable against and removably attached to a section of the body member 11. As used herein, the term “section” is used in conjunction with the orthotic device 10 of the present invention and refers to the major areas of the body member 11 (e.g., upper section 12 and lower section 13). The upper section 12 and lower section 13 of the body member 11 are delineated at the bend in the body member as depicted in FIGS. 9, 10A, and 10B. It will be understood, however, that a section does not necessarily imply an area of the body member 11 that is separate from the other areas of the body member. Stated differently, it will be understood that a section may be a separate or integral component of the body member 11. The term “portion” is used in conjunction with the orthotic device 10 of the present invention and refers to the major areas of the flexible sheet member 15 (e.g., upper portion and lower portion). It will be understood, however, that a “portion” does not necessarily imply an area of the sheet member 15 that is separate from the other areas of the sheet member. Stated differently, it will be understood that a portion may be a separate or integral component of the sheet member 15. For example, one embodiment of the sheet member 15 may be formed from a single piece of material. Thus, a portion may refer to an area of the sheet member 15 that is defined by a part of the single piece of material forming the sheet member 15. That said, it will also be understood that the sheet member 15 may be formed from multiple pieces of material that are secured to one another to form the sheet member 15. In this case, a portion may be a separate piece of material forming the sheet member 15. The term “secured” may refer to sewn, made integral with, adhered with adhesive, or bonded with heat.

As illustrated in FIGS. 1 and 3, the upper section 12 of the body member 11 engages an anterior portion of a lower leg and the lower section 13 of the body member 11 engages a dorsal portion of a foot. It will be understood by those skilled in the art that the terms “upper section” and “lower section”, and “upper portion” and “lower portion”, may also refer to “lower section” and “upper section”, and “lower portion” and “upper portion”, respectively, dependent upon the perspective of the individual viewing the apparatus. It will also be appreciated that the term “upper” implies the opposite of “lower.”

The body member 11 has an external surface 20 and an internal surface 21. With reference to the orientation of the brace in FIG. 9, it will be understood by those of skill in the art that the terms “internal surface” and “external surface” may be referred to as “inside surface” and “outside surface.” Stated differently, as used herein the term internal surface implies the side of the apparatus closest to the ankle or foot of the wearer. Thus, it will be understood that the term external surface implies the side of the apparatus opposite the internal surface (i.e., the side farthest from the ankle or foot of the wearer).

The external surface 20 of the body member 11 may include fasteners 22 for removably attaching a portion of the flexible sheet member 15 to a portion of the external surface 20 of the body member 11. With respect to the term “fasteners”, although hook-and-loop fasteners are illustrated, it should be recognized that other fasteners could be used, if desired, although not necessarily with equivalent results. Fasteners include a wide variety of devices for securing the components of the orthotic device 10. Thus, a fastener includes a hook-and-loop fastener, snaps, lace and strings, or any other element or combination of elements capable of securing various components, alone or in combination with other fastener elements, to other components of the orthotic device 10.

In one novel aspect of the present invention, the stabilizing member 14 is adjustable (i.e., bendable) and fixed to portions of the upper and lower sections 12, 13 of the body member 11. In one embodiment, the stabilizing member 14 is elongate and extends the length of a portion of the upper and lower sections 12, 13 of the body member 11. As depicted in FIGS. 10A and 10B, a user may manually adjust the position of the upper section 12 of the body member 11 relative to the lower section 13 of the body member dependent upon the desired angle (e.g., a or a′) of support for the foot relative to the lower leg. By doing so, the device promotes healing of the plantar fascia and reduces the pain associated therewith. The stabilizing member 14 may be formed from metal, alloy or any number of materials providing sufficient rigidity and adequate adjustability for wear on the foot (e.g., aluminum). Adjustment provided by, for example, an aluminum stabilizing member 14, is accomplished without additional mechanical devices (e.g., a locking slide) or heat requirements (e.g., heating and molding the body member 11).

Accordingly, the device provides a novel combination of a body member 11, sheet member 15, and a stabilizing member 14, wherein the stabilizing member is manually adjustable yet sufficiently rigid to resist movement during wear. Stated differently and referring to FIGS. 10A and 10B, the stabilizing member 14 is sufficiently rigid to maintain a desired angle a or a′ between the upper section 12 and lower section 13 of the body member 11 during wear. It will be understood that as used herein, the concept of a component (e.g., padding) being “between” two other components does not necessarily imply that the three components are contiguous (i.e., in intimate contact). Rather, as used herein, the concept of one component being between two other components is meant to describe the relative positions of the components within the orthotic device, respectively.

In practice, the flexible sheet member 15 adjustably secures the body member 11 against the lower leg and foot as illustrated in FIGS. 1-3. Referring to FIG. 9, one embodiment of the invention includes a flexible sheet member 15 having at least one portion fixed to the body member 11 and at least another portion removably attachable to the body member. In particular, one embodiment of the orthotic device shown in FIG. 2 includes a flexible sheet member 15 having at least one portion fixed to an edge of the upper section 12 of the body member 11, and at least another portion removably attachable to the lower section 13 of the body member. With reference to FIG. 9, fasteners 22 are provided on the flexible sheet member 15 for removably attaching portions thereof to the body member 11.

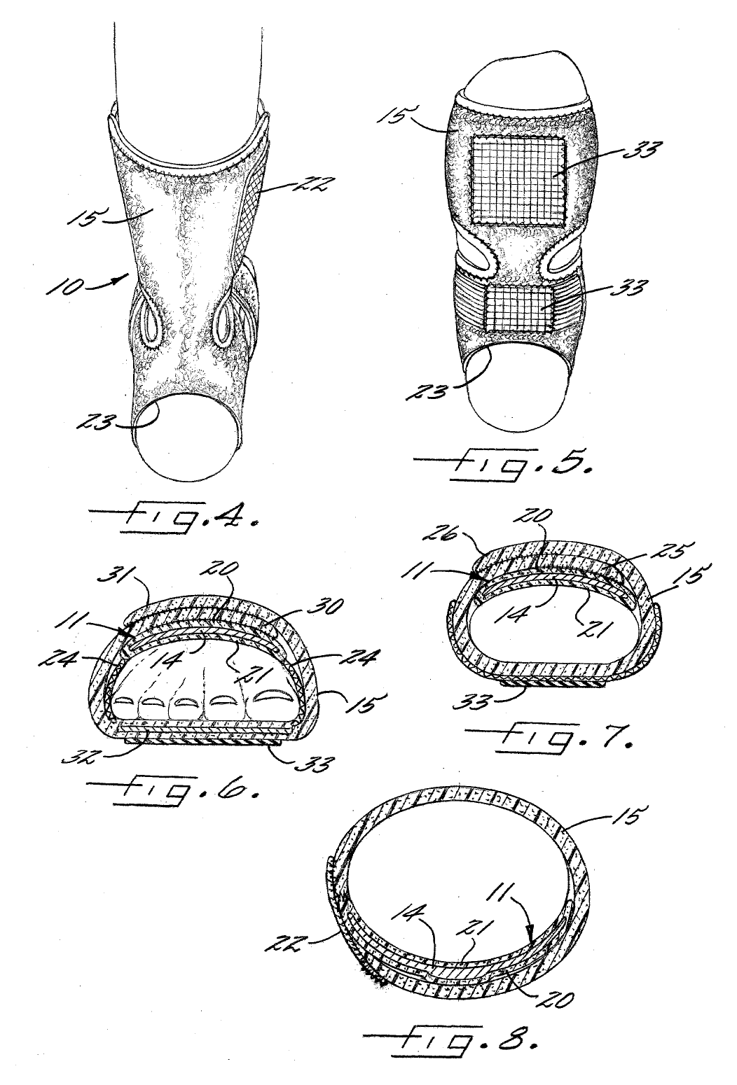

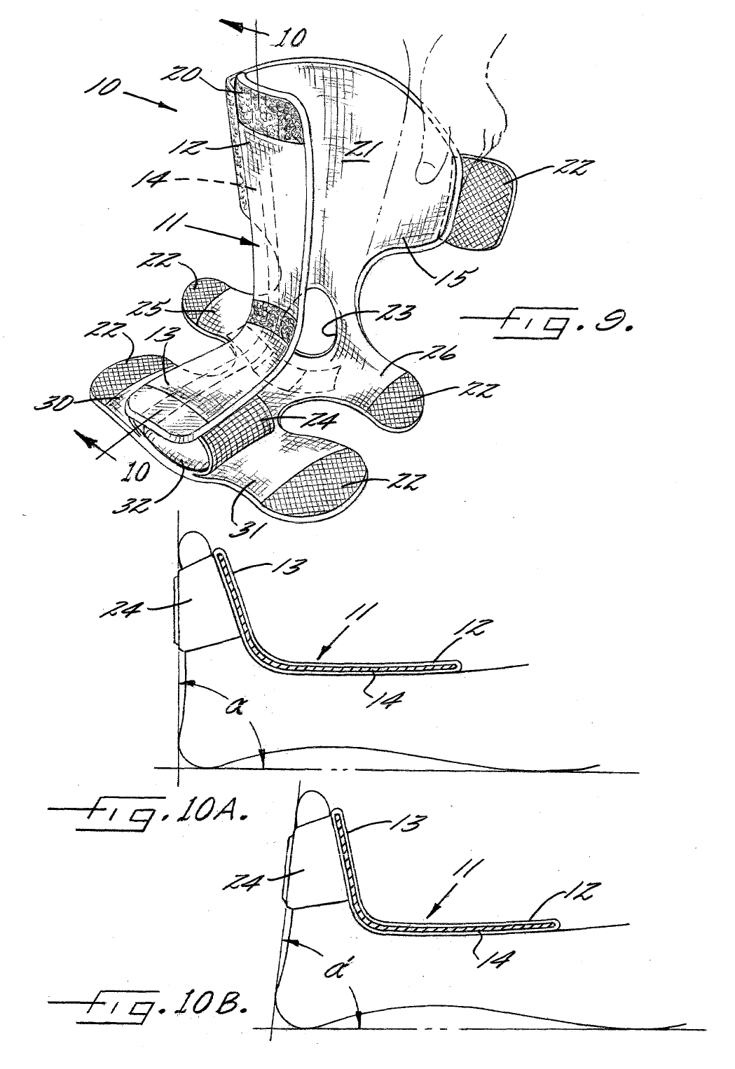

Referring to FIGS. 4 and 9, the flexible sheet member 15 defines at least one opening 23 for receiving portions of a heel during use. The flexible sheet member 15 may be formed from an elastomeric material that promotes adjustability yet provides sufficient support and comfort. As referenced herein, the term “elastomeric” describes a material having characteristics of various elastic substances resembling rubber. As used herein, it will be understood that the term “elastic” refers to material that is capable of being easily stretched or expanded and resuming its former shape. Stated differently, the term elastic implies the property of resisting deformation by stretching. In a related aspect, it will be understood by those skilled in the art that the term “inelastic” refers to material that resists stretching and elongation.

Referring to FIG. 9, one embodiment of the orthotic device includes at least one flexible panel 24 or a pair of flexible panels 24 that connect the flexible sheet member 15 to the lower section 13 of the body member 11. In particular, the pair of flexible panels 24 connects lower portions of the flexible sheet member 15 to edges of the lower section 13 of the body member 11. In use, the pair of flexible panels 24 position lower portions of the flexible sheet member 15 substantially adjacent to or against a lower portion of the foot. Those skilled in the art will appreciate that the term “substantially adjacent” refers to two or more components that have a common border or are in close proximity to one another. Nevertheless, it will be understood that adjacent may or may not imply contact, but always implies the absence of anything of the same kind in between.

Referring to FIG. 9, the orthotic device of the invention also provides a first and second rear (or heel) tensioning arms 25, 26 positioned at sides of the opening 23 in the flexible sheet member 15. Stated differently, the invention may provide a pair of heel tensioning arms 25, 26. Fasteners 22 are provided at ends of each heel tensioning arm 25, 26 such that a user may removably attach the heel tensioning arms 25, 26 and a portion of the flexible sheet member 15 to the body member 11 as illustrated in FIGS. 3 and 9.

In more detail, the first heel tensioning arm 25 is positioned at one side of the opening 23 in the flexible sheet member 15 and the second heel tensioning arm 26 is positioned at an opposite side of the opening 23. In other words, a pair of rear or heel tensioning arms 25, 26 is positioned at opposing sides of the opening 23 in the flexible sheet member 15.

Still referring to FIG. 9, the orthotic device of the invention further provides a first and second front (or foot) tensioning arms 30, 31 positioned at sides of the flexible sheet member 15. In other words, the orthotic device may include a pair of front or foot tensioning arms 30, 31. The orthotic device 10 also provides fasteners 22 for removably attaching the foot tensioning arms 30, 31 and at least a portion of the flexible sheet member 15 to the body member 11.

As illustrated in FIGS. 6 and 9, the first foot tensioning arm 30 is positioned at one side of the flexible sheet member 15, and the second foot tensioning arm 31 is positioned at an opposite side of the flexible sheet member 15. FIG. 6 depicts the pair of foot tensioning arms 30, 31 positioned at opposite sides of a lower portion of the flexible sheet member 15. The adjustable support provided by the heel and foot tensioning arms 25, 25, 30, 31, in combination with the flexible nature of the sheet member 15, provides improved support and adjustability for the user.

The orthotic device further provides a base pad 32 fixed to a lower portion of the flexible sheet member 15. The base pad 32 engages a lower portion of the foot as illustrated in FIGS. 5 and 9. As configured, the base pad 32 is positioned substantially adjacent to the lower section 13 of the body member 11. FIGS. 6 and 9 depict the base pad 32 on the lower section 13 of the sheet member 15 and substantially adjacent to the flexible panels 24. The flexible panels 24 connect the lower portion of the sheet member 15 at opposing sides of the base pad 32.

Referring to FIG. 5, the invention includes one or more non-slip pads 33 secured to lower portions of the flexible sheet member 15. In particular, the non-slip pads 33 are secured to the sheet member 15 near the heel and ball of the foot. Thus, at least one non-slip pad 33 is provided on the external surface of the sheet member 15 substantially opposite the base pad 32. During use, a substantial amount of force is directed from the base pad 32 to at least one non-slip pad 33 to prevent a user from slipping on smooth surfaces during limited activity.

Accordingly, a user may adjustably secure the orthotic device 10 to the leg and foot. Specifically, the user places a front portion of the foot between the lower section 13 of the body member 11 and the base pad 32 such that the flexible panels 24 draw the body member against the anterior portion of the lower leg and the dorsal portion of the foot. Next, the user applies the flexible sheet member 15 against the lower leg and foot such that the opening 23 in the sheet member 15 receives the heel, and the flexible sheet member surrounds the posterior portions of the lower leg. Subsequently, the user releasably secures the flexible sheet member 15 to the exterior surface of the body member 11 with fasteners 22 provided. During adjustment, the user draws the foot tensioning arms 30, 31 upward and against the external surface 20 of the lower section 13 of the body member 11, and releasably secures the foot tensioning arms 30, 31 to the body member 11 at a desired amount of support. In addition, the user may draw the heel tensioning arms 25, 26 against the body member 11 and secure the arms thereto at a desired amount of support. Before or after initial fitment of the orthotic device 10 to the leg and foot, the user may manually adjust the stabilizing member 14 to achieve a desired angle of support (e.g., a or a′) as depicted in FIGS. 10A and 10B. Thus, the orthotic device 10 promotes healing of the plantar fascia by facilitating the manual adjustment of the stabilizing member 14 and body member 11 to achieve a desired angle of support of the foot relative to the leg during wear.

In the drawings and specification, there have been disclosed typical embodiments on the invention and, although specific terms have been employed, they have been used in a generic and descriptive sense only and not for purposes of limitation, the scope of the invention being set forth in the following claims.

That which is claimed is:

1. An orthotic device for a lower leg comprising:
a body member having an upper section and a lower section;
an adjustable stabilizing member fixed to said body member, said adjustable stabilizing member extending the length of at least a portion of said upper section and said lower section of said body member; and
a flexible sheet member having at least a portion that is foldable against and removably attached to at least a portion of said body member, said flexible sheet member defining at least one opening.

2. An orthotic device according to claim 1 wherein said body member has an external surface and an internal surface, said external surface having fasteners for removably attaching at least a portion of said flexible sheet member to at least a portion of said external surface of said body member.

3. An orthotic device according to claim 1 wherein said flexible sheet member is formed from an elastomeric material.

4. An orthotic device according to claim 1 further comprising a flexible panel securing a portion of said flexible sheet member to said lower section of said body member.

5. An orthotic device according to claim 1 further comprising a tensioning arm positioned at one side of said opening in said flexible sheet member, said tensioning arm removably attachable to said body member.

6. An orthotic device according to claim 1 further comprising a tensioning arm positioned at one side of said flexible sheet member, said tensioning arm removably attachable to said body member.

7. An orthotic device according to claim 1 further comprising a base pad fixed to a lower portion of said flexible sheet member and positioned substantially adjacent to said lower section of said body member.

8. An orthotic device for a lower leg comprising:
a body member having an upper section for engaging an anterior portion of a lower leg, and a lower section for engaging a dorsal portion of a foot;
a manually adjustable stabilizing member fixed to portions of said upper section and said lower section of said body member; and
a flexible sheet member for adjustably securing said body member against the lower leg and foot, said flexible sheet member having one portion fixed to said body member and having at least another portion removably attachable to said body member, said flexible sheet member defining at least one opening for receiving portions of a heel.

9. An orthotic device according to claim 8 wherein said body member having fasteners for removably attaching said at least another portion of said flexible sheet member to said body member.

10. An orthotic device according to claim 8 wherein said stabilizing member is sufficiently rigid to resist movement during wear.

11. An orthotic device according to claim 8 further comprising:
a first heel tensioning arm positioned at one side of said opening in said flexible sheet member, said first heel tensioning arm removably attachable to said body member; and
a second heel tensioning arm positioned at an opposite side of said opening in said flexible sheet member, said second heel tensioning arm removably attachable to said body member.

12. An orthotic device according to claim 11 wherein said first and second heel tensioning arms having fasteners for removably attaching at least a portion of said flexible sheet member to said body member.

13. An orthotic device according to claim 8 further comprising:
a first foot tensioning arm positioned at one side of said flexible sheet member, said first foot tensioning arm removably attachable to said body member; and
a second foot tensioning arm positioned at an opposite side of said flexible sheet member, said second foot tensioning arm removably attachable to said body member.

14. An orthotic device according to claim 13 wherein said first and second foot tensioning arms having fasteners for removably attaching at least a portion of said flexible sheet member to said body member.

15. An orthotic device according to claim 8 further comprising a pair of flexible panels for positioning said flexible sheet member against a lower portion of the foot, said pair of flexible panels connecting said flexible sheet member to said lower section of said body member.

16. An orthotic device according to claim 8 further comprising a base pad for engaging a lower portion of the foot, said base pad fixed to a lower portion of said flexible sheet member and positioned substantially adjacent to said lower section of said body member.

17. An orthotic device for a lower leg comprising:
a body member having an upper section for engaging an anterior portion of a lower leg, and a lower section for engaging a dorsal portion of a foot;
an elongate stabilizing member fixed to said body member and extending the length of at least a portion of said upper section and said lower section of said body member;
a flexible sheet member for adjustably securing said body member against the lower leg and foot, said flexible sheet member having one portion fixed to said body member and having other portions removably attachable to said body member, said flexible sheet member defining at least one opening for receiving portions of a heel;
a pair of rear tensioning arms positioned at opposing sides of said opening in said flexible sheet member, said pair of rear tensioning arms removably attachable to said body member; and
a pair of front tensioning arms positioned at opposite sides of a lower portion of said flexible sheet member, said pair of front tensioning arms removably attachable to said body member.

18. An orthotic device according to claim 17 wherein said flexible sheet member having fasteners for removably attaching said other portions of said flexible sheet member to said body member.

19. An orthotic device according to claim 17 wherein said stabilizing member is adjustable, yet sufficiently rigid to maintain a desired angle between said upper section and said lower section of said body member during wear.

20. An orthotic device according to claim 17 wherein said flexible sheet member has at least one portion fixed to an edge of said upper section of said body member, and at least another portion removably attachable to said lower section of said body member.

21. An orthotic device according to claim 17 wherein said pair of rear tensioning arms and said pair of front tensioning arms having fasteners for removably attaching at least a portion of said flexible sheet member to said body member.

22. An orthotic device according to claim 17 further comprising a pair of flexible panels for positioning said flexible sheet member against a lower portion of the foot, said pair of flexible panels connecting lower portions of said flexible sheet member to edges of said lower section of said body member.

23. An orthotic device according to claim 17 further comprising a base pad for engaging a lower portion of the foot, said base pad fixed to a lower portion of said flexible sheet member and positioned substantially adjacent to said lower section of said body member.
